(19) United States
(12) Patent Application Publication
(10) Pub. No.: US 2003/0068383 A1
(43) Pub. Date: Apr. 10, 2003

(54) Time release nutritional supplement

(76) Inventors: James A. Patterson, Sarasota, FL (US); Douglas R. Goodman, Sarasota, FL (US)

Correspondence Address: Charles J. Prescott, Charles J. Prescott, P.A., Suite 115, 2033 Wood Street, Sarasota, FL, 34237-7926, US

(21) Appl. No.: 09/969,454

(22) Filed: Oct. 2, 2001

(51) Int. Cl. A61K 9/58; A61K 35/78

(52) U.S. Cl. 424/501; 424/725

(57) ABSTRACT

A time-release nutritional dietary supplement in a pharmaceutically acceptable vehicle for ingestion. The nutritional supplement is held or bound within the cross-linked structure of hydrophilic resin particles or beads. The selection of percent cross-linking of the resin particles and the selected quantity of the nutritional supplement loaded into that cross-linked structure will determine the time period over which the nutritional supplement is released after ingestion. Embodiments of this invention may include acid-sensitive and acid-insensitive nutrient supplements. Prior to mixture loading of an acid-sensitive supplement into the resin particles, the resin may be ion exchanged with a soluble monovalent salt formed, for example, of Li or K which enables the modified resin particles to be loaded with the acid-sensitive nutritional supplement without damage.

### BACKGROUND OF THE INVENTION

1. Scope of Invention

This invention relates generally to time-release mechanisms for ingested bioactive agents and more particularly to the prolonged time release of nutritional supplements.

2. Prior Art

Mechanisms for the time release of nutrients ingested into human or living systems have grown since the mid 1960s. Mechanisms such as those which were erodeable and wherein the bioactive agent absorbed into the erodeable matrix is released as it is eroded and dissolved within the digestive system represented an early approach to this need.

Other methods of nutrient and medication time release include the encapsulation of the bioactive agent within a polymeric shell through which the agent defuses. The rate of diffusion controls the rate of release of the bioactive agent in the digestive system.

An aspect of the present invention particularly focuses upon the time release of creatine within the human body. Creatine is known to have beneficial effects as a dietary supplement. Once creatine is in muscle, it serves as a reservoir of high potential phospheral groups. Creatine is available from many food sources and can be found in the muscles of most mammals and fish, including beef, chicken, cod, herring, pork, salmon, tuna and turkey. Because of the high fat content of this natural source of creatine, creatine supplements have become a popular source of obtaining the nutrient.

Creatine has also been used in increasing body mass, strength and energy and for reducing body fat. Once creatine enters the muscle fibers, it accumulates and stays there for several weeks. However, the ingestion of large quantities of creatine is not practical as there is no convenient way to meter or control the rate of absorption into the human fibers and digestive system without causing substantial side effects. Doses of over 5 grams are typically associated with side effects that include diarrhea, gas, flatulence, nausea and stomach cramps.

In general terms, there is a need for an improved bioinactive and insoluble matrix or excepient for the time release of nutritional supplements into the human body and animals which will slowly deliver the beneficial bioactive agents into the system on a controlled and predictable timed basis and without causing any severe side effects either through excessive introduction of the bioagents into the system or the disposition of the insoluble carrier or matrix into which the bioactive agents have been placed.

### BRIEF SUMMARY OF THE INVENTION

This invention is directed to a time-release nutritional dietary supplement in a pharmaceutically acceptable vehicle for ingestion. The nutritional supplement is held or bound within the cross-linked structure of hydrophilic resin particles or beads. The selection of percent cross-linking of the resin particles and the selected quantity of the nutritional supplement loaded into that cross-linked structure will determine the time period over which the nutritional supplement is released after ingestion, the concept being to delay nutrient dispersion until it reaches the small intestine. Prior to mixture loading of an acid-sensitive supplement into the resin particles, the resin may be ion exchanged with a soluble monovalent salt formed, for example, of Li or K which enables the modified resin particles to be loaded with an acid-sensitive supplement nutritional and acid-sensitive supplements without damage.

It is therefore an object of this invention to provide a time-release nutritional supplement which will provide a predictable time release of a broad array bioactive nutrients or nutritional supplements and which will be easily processed through the digestive system as the nutrients are disbursed on a time-release basis.

It is another object of this invention to provide a time release mechanism which, when appropriately modified at preparation, will provide a time release carrier for vitamins, minerals, botanicals, carbohydrates and amino acids in their various forms.

It is still another object of this invention to provide a time release creatine which will allow for the ingestion of large quantities of creatine in a single dose which will meter the creatine into the digestive system and muscle tissue at a rate which will not cause severe side effects and which will be usefully absorbed into the muscle tissue by the time release mechanism.

Still another object of this invention is to provide a time-release mechanism which will substantially inhibit digestion of the nutritional supplement until it reaches the small intestine by enhanced resistance to stomach acid.

In accordance with these and other objects which will become apparent hereinafter, the instant invention will now be described with reference to the accompanying drawings.

### DETAILED DESCRIPTION OF THE INVENTION

### Broad Concept

Resin Matrix

The broad aspect of the present invention embodies using a polymer resin in small particle or bead form which is cross linked to provide nutrient supplement absorption into the matrix created at the formation of the resin. Preferably, the resin is of a cross linked nature and may preferably be cross-linked up to ten percent (10%) and in excess of that level of cross-linking as well. However, the preferred limitation of 10% cross linking provides a minimally sufficient amount of nutrient supplement absorption into the matrix structure for later dispersion therefrom once the product has been ingested. The osmotic forces required to separate the nutritional supplement from higher cross-linked resin becomes excessive.

Cross linked resin particles are provided in two forms. In order for the resin to absorb nutrient supplements, it must be rendered hydrophilic. To accomplish this, the resin is sulfonated after it has been produced. The sulfonated form of the resin or excepient is preferably used with nutrient supplements which are generally characterized as being acid insensitive. That is to say that the nutrient supplement will not substantially degrade in the presence of an acid which is produced when the sulfonated resin is combined in accordance with the teachings herebelow. Where nutrient supplements chosen for being loaded into the porous resin matrix are of an acid-sensitive nature such as sugars which freely hydrolyze, the resin is ion exchanged with a soluble monovalent salt such as lithium or potassium salt.

An additional limitation with respect to the percentage of cross linking is the increasing resistance to the hydrophilic expansion of the resin when heated and placed in the mixed presence of a heated liquefied nutrient supplement. The greater the degree of cross-linking, the more resistant the matrix structure exhibits to expansion and therefore absorption of the liquid nutrient supplement. Moreover, the osmotic force required to remove nutrient supplements from higher cross-linked resin becomes excessive to the point where the nutrient supplement will not be timely removed for absorption into the small intestine.

At the extreme lower end of the percentage cross linking, the resin, when cross linked at below 0.5% cross linking will produce a micro sponge-like structure which is quite unstable and difficult to work with when the nutrient supplement is to be absorbed into this micro sponge structure.

Addition of Heat

To enhance the take-up of nutrient supplements into the porous excepient matrix, the nutrients are preferably first dissolved in liquid which has been heated so as to maximize the saturation of the nutrient supplement within the heated liquid. Therefore, each unit of heated liquid absorbed into the matrix structure results in a corresponding increase in the amount of nutrient supplements held within the matrix once the combined structure is cooled and partially dry.

Moreover, the actual heating of the resin matrix prior to combination with the heated nutrient supplement bearing liquid substantially enhances the uptake of the amount of nutrient supplement laden liquid. The preheating of the resin up to about 105°-110° C. dries and expands the porous resin structure to cause a substantial increase in the nutrient supplement loading capacity of the resin.

Resin Particle Size

Control of the resin particle size has an inverse affect upon the effectiveness of the resin to accomplish its task of maximizing nutritional supplement take-up as well as to facilitate the removal of the nutritional supplement after ingestion. The preferred range of resin particle size is up to about 200 mesh (dry). However, the preferred resin particle size is in the range of 25 to 100 microns.

Nutrient Supplements

The present invention is particularly adapted to supportively receive a broad range of nutrient supplements. These include by general category the following which are described in more detail herebelow:

I AMINO ACIDS

II VITAMINS

III MINERALS

IV BOTANICALS

V CARBOHYDRATES

The following tables provide a more detailed description of the various nutrient supplements which are particularly adapted to the present invention.

TABLE 1
AMINO ACIDS

| BRANCHED CHAIN | ESSENTIAL AMINO ACIDS |
|---|---|
| Isoleucine | Tryptophan |
| Leucine | Lysine |
| Valine | Methionine |
| | Phenylalaine |
| | Treonine |
| | Valine |
| | Leucine |
| | Isolleucine |
| | AMINA ACID LIKE |
| NON-ESSENTIAL AMINO ACIDS | NEUTRACEUTICALS |
| Arginine | Creatine Monohydrate |
| Tyrosine | L-Carnatine |
| Glycine | Calcium Pyruvate |
| Serine | Adenosine 5 Monophosphate |
| Glutamic Acid | Beta-Hydroxy Beta-Methlbutyrate |
| Aspartic Acid | |
| Taurine | |
| Cystine | |
| Proline | |
| Alanine | |
| Histidine | |

TABLE II
VITAMINS

| ANTIOXIDANTS | FAT SOLUBLE |
|---|---|
| A - Beta Carotene | A - Beta Carotene |
| C - Ascorbic Acid | D - Ergcalciferol |
| E - Acetate | E - Acetate |
| Selenium (A mineral) | K - |
| WATER SOLUBLE | |
| B1 - Thiamine | |
| B2 - Riboflavine | |
| B3 - Nicotinamide | |
| B5 - Panthenol | |
| B6 - Pyrodoxine | |
| B8 - Folate | |
| B12 - Cyanocobalamin | |
| Biotine | |
| C - Ascorbic Acid | |

TABLE III
MINERALS

| MACRO MINERALS | ELECTROLYTE MINERALS |
|---|---|
| Calcium | Sodium |
| Magnesium | Chloride |
| Phosphorus | Potassium |
| Sulfur | |
| TRACE MINERALS | |
| Boron | |
| Chromium | |
| Copper | |
| Iodine | |
| Irone | |
| Magnesium | |
| Molybedenum | |
| Selenium | |
| Zinc | |

TABLE IV
BOTANICALS

Ciwajia Siberian Ginseng
Gingko Biloba
Guarana Extract
Maitake Mushroom

TABLE V
CARBOHYDRATES

D-Ribose
D-Glucose

Preparing the Resin

A 1.0% cross-linked polystyrene divinyl benzene spherical polymer (200 mesh 100μ) was washed and dried. 10.7 grams of the polymer was placed in a 1 liter glass beaker. To this was added excess methylene chloride (Met cl), a swelling agent for the polyer for ½ hour to complete the swelling of the polymer. After this time, 220 ml. of chloride sulfonic acid was added slowly to avoid overheating the methylene chloride which boils at 50° C. The mixed acid-polymer reaction time was 1.0 hour. The sulfonated polymer is thereby converted from a hydrophobic to a hydrophilic polymer.

The sulfonated polymer was then hydrated using a small stream of H₂O (DI) until the resin sank. Then, some of the was decanted off top layer, the resin then washed with more DI H₂O. The lost DI water was retained and 1% by volume H₂O₂ is added and the mixture is stirred with a magnetic stirer and heated to 60° to 80° C. for 0.5 hours.

The cool resin was then placed on a glass fretted filter (course) and vacuum was applied to remove excess liquid and rewashed about three times to remove all acid to a pH of 5-6. This dry polymer was weighed and a a weight adequate portion was taken for a sample. This weighed sample is oven dried to determine weight losses on overnight drying at 110° C.

### EXAMPLES

### Example 1

To demonstrate the need for eliminating the possibility of acid damage to a nutritional supplement, if 2 grams of 1% cross-linked resin are combined with 5.9 grams of Ribose and 10 grams of water preheated to boiling, the Ribose will be destroyed within the sulfonated resin matrix.

However, if the sulfonated resin has been ion exchanged with one of the above-referenced salts, the combination of 2 grams of the ion exchanged resin with the 5.9 grams of Ribose and 10 grams of water will produce a matrix supporting active and useful Ribose absorbed therein.

### Example 2

Creatine has shown to be of particular interest to applicants with respect to performance enhancement. One example of creatine in combination with the resin support matrix or excepient is as follows:

26 g of creatine are combined with 150 ml of water held at boiling temperature. Heated water/creatine liquid is then added to 10 g of dry 1% cross-linked and sulfonated resin. The mixture is dried sufficiently to be loaded into capsules as a free flowing powder. Each capsule will be loaded with 0.9 g of this partially dried powder mixture.

The total mixture after partial drying will weight approximately 58 g thus indicating that 23 g of the 150 g of water are left in the mixture which is characterized as free flowing but “mushy” in physical character.

While the instant invention has been shown and described herein in what are conceived to be the most practical and preferred embodiments, it is recognized that departures may be made therefrom within the scope of the invention, which is therefore not to be limited to the details disclosed herein, but is to be afforded the full scope of the claims so as to embrace any and all equivalent apparatus and articles.

What is claimed is:

1. A time-release nutritional supplement comprising:
a quantity of a nutritional supplement in powder form taken from the group consisting of amino acids, carbohydrates, vitamins and botanical herbs combined within an excepient in the form of insoluble cross-linked structure of resin particles.

2. A time-release nutritional supplement comprising:
a quantity of a soluble nutritional supplement in powder form taken from the group consisting of amino acids, carbohydrates, vitamins and botanical herbs bound within insoluble resin particles having a cross-linked structure of up to about 10% cross linking;
said nutritional supplement and said resin particles in a pharmaceutical acceptable vehicle for internal ingestion.

3. A time-release nutritional supplement as set forth in claim 2, wherein:
said cross-linking is at least about 1% cross-linking of said resin.

4. A time-release nutritional supplement comprising:
a quantity of hydrophilic nutritional supplement in substantially dry powder form taken from the group consisting of amino acids, carbohydrates, vitamins and botanical herbs bound within sulfonated hydrophilic resin whereby release of said nutritional supplement from said resin after ingestion is timed.

5. A time-release nutritional supplement comprising:
a quantity of nutritional supplement in powder form taken from the group consisting of amino acids, carbohydrates, vitamins and botanical herbs bound or held within cross-linked and sulfonated styrene divinyl benzene resin particles which have been ion exchanged with a soluble monovalent salt;
said nutritional supplement of the type which is acid-sensitive and remaining substantially unchanged by combination with said resin particles;
whereby release of said nutritional supplement occurs over a predetermined time period;
said nutritional supplement and said resin particles in a pharmaceutical acceptable vehicle for ingestion.

6. A time-release nutritional supplement as set forth in claim 5, wherein:
said monovalent salt is formed of Li or K.

7. A time-release nutritional supplement as set forth in claim 5, wherein:
said cross-linking is at least about 1%.

8. A time-release nutritional supplement as set forth in claim 5, wherein:
said resin is formed as a microsponge and said cross-linking is substantially less than 1%.

9. A time-release nutritional supplement in powder form taken from the group consisting of amino acids, carbohydrates, vitamins and botanical herbs comprising:
a quantity of said nutritional supplement bound or held within cross-linked and sulfonated styrene divinyl benzene resin particles;
said nutritional supplement of the type which is acid-insensitive;
whereby release of said nutritional supplement occurs over a predetermined time period;
said nutritional supplement and said resin particles in a pharmaceutical acceptable vehicle for ingestion.

10. A time-release nutritional supplement as set forth in claim 9, wherein:
said cross-linking is at least about 1%.

11. A time-release nutritional supplement as set forth in claim 9, wherein:
said resin is formed as a microsponge and said cross-linking is substantially less than 1%.
